(19) United States
(12) Patent Application Publication
(10) Pub. No.: US 2005/0049550 A1
(43) Pub. Date: Mar. 3, 2005

(54) Mixing device for multiple-chamber ampoule

(76) Inventors: Fritz Kirchhofer, Sumiswald (CH); Christoph Rindlisbacher, Boll (CH)

Correspondence Address: David E. Bruhn, Esq.;DORSEY & WHITNEY LLP, Intellectual Property Department, 50 South Sixth Street, Suite 1500, Minneapolis, MN, 55402-1498, US

(21) Appl. No.: 10/927,623

(22) Filed: Aug. 26, 2004

(30) Foreign Application Priority Data
Sep. 3, 2003 (DE) DE 103 40 586.0

(51) Int. Cl. A61M 37/00

(52) U.S. Cl. 604/82; 604/232

(57) ABSTRACT

A mixing device for an injectable product including a casing, an ampoule including at least a first chamber containing a first product component and at least a second chamber containing a second product component, and a drive mechanism which drives a stopper in the ampoule relative to the casing using a translational movement.

### BACKGROUND

The present invention relates to devices and methods for administering, injecting or dispensing substances, including medicinal substances. More particularly, it relates to a device and method for mixing an injectable product in a multiple-chamber ampoule. In one embodiment, the invention relates to a mixing device for an administering device, such as for instance an injection pen or the like, which is suitable for administering an injectable product from a multiple-chamber ampoule. This application claims the priority of German patent application No. 103 40 586.0-44, filed on Sep. 3, 2003 with the German Patent and Trademark Office.

In various medical or therapeutic procedures, fluid products are administered to a patient which consist of a number of components which are not mixed until shortly before being administered. Growth hormones, for example, are one such fluid product. In order to administer such fluid products, multiple-chamber ampoules—in particular, bicameral ampoules—are used, in which a liquid is provided as a solvent in a first chamber and a product component is provided in a solid or also liquid state in a second chamber. The solvent and the product component are mixed in the ampoule by means of a mixing device. With the aid of the mixing device, the fluid product to be injected can be mixed shortly before it is administered by an administering device, by shifting a stopper within the multiple-chamber ampoule such that the solvent comes into contact with the product component via a supply channel and mixes with it. One example of a multi-chamber ampoule and/or injection device is disclosed in U.S. Pat. No. 5,728,075, the disclosure and teachings of which are incorporated herein by reference. Other examples of such ampoules and injection devices, some of which may have more than two chambers, are known to those skilled in the art.

When mixing the fluid product, care must be taken that the mixing procedure is not performed too rapidly. Rapid mixing can, for example, lead to a foam undesirably forming in the fluid product. Furthermore, if rapidly mixed, it is possible that a solid product is not yet completely dissolved in a solvent by the time it is administered or that the solution of the fluid product is not yet homogenous.

In a conventional administering device, the multiple-chamber ampoule is inserted into a casing of the device. The administering device comprises a mixing device in which a drive mechanism can be screwed into the casing with the aid of a thread. The drive mechanism is screwed into the casing by hand using a rotational movement, wherein it moves relative to and into the casing. Rotating in the drive mechanism of the mixing device slowly moves the stopper within the multiple-chamber ampoule, axially along the ampoule. Accordingly, the rotational movement of the drive mechanism is converted into a slow translational movement of the stopper within the ampoule, such that it is possible to gently mix the product within the ampoule. Rotating in the drive mechanism in order to mix the injectable product is, however, an awkward and elaborate procedure.

### SUMMARY

It is an object of the present invention to provide a mixing device for an injectable product in a multiple-chamber ampoule, which is simple to operate and in which the product is reliably mixed, in particular not too rapidly.

The object is addressed by providing, in accordance with the present invention, a mixing device for mixing an injectable product, comprising a casing and a bicameral ampoule which comprises at least a first chamber with a first product component and at least a second chamber with a second product component, wherein the mixing device further comprises a drive mechanism which drives a stopper in the bicameral ampoule using a translational movement.

In one embodiment, the present invention comprises a mixing device for an injectable product including a casing, an ampoule including at least a first chamber containing a first product component and at least a second chamber containing a second product component, and a drive mechanism which drives a stopper in the ampoule relative to the casing using a translational movement.

A device for mixing an injectable product accordingly comprises a casing and a multiple-chamber ampoule. The casing can exhibit a cylindrical shape. Other means for administering the product, such as a dosing mechanism, or a dispensing mechanism, can be accommodated in the casing. The multiple-chamber ampoule comprises at least a first chamber with a first product component, such as a liquid solvent, and a second chamber with a second product component, such as a solid. In the following description, bicameral ampoules are considered, such as those already used for growth hormones. The mixing device in accordance with the invention is, however, also suitable for ampoules with more than two chambers.

The bicameral ampoule can be inserted into the casing and can, in some embodiments, be fixedly connected to it. After mixing, the bicameral ampoule is available for administering the product or can be removed from the casing. The mixing device is preferably reusable, such that a new bicameral ampoule can be inserted into the casing and the product administered from it. In accordance with the invention, the mixing device for mixing the first and second product component in the bicameral ampoule comprises a drive mechanism which drives a stopper in the bicameral ampoule using a translational movement. Due to the translational movement, the drive mechanism exerts a drive force on the stopper in the bicameral ampoule, such that the stopper is moved with respect to the casing of the mixing device, in the axial direction with respect to the casing and/or the bicameral ampoule. The translational movement of the drive mechanism is preferably performed automatically. The translational movement of the drive mechanism can be started in order to automatically mix the product, e.g., by inserting the bicameral ampoule into the casing of the mixing device, or an independent trigger for triggering the translational movement of the drive mechanism can be provided on the mixing device.

In some preferred embodiments, a biasing mechanism for biasing the drive mechanism before inserting the bicameral ampoule is provided in the mixing device. In this way, shortly before the mixing device is used and the injectable product mixed, the mixing device is placed in a biased state which enables a drive force to be exerted on the stopper of the ampoule by the drive mechanism after or when the bicameral ampoule has been inserted. In order to secure the mixing device in this biased position, a latching mechanism for latching the drive mechanism is provided. When the bicameral ampoule has been inserted, the latching mechanism's latching of the drive mechanism is released by a trigger and the biasing force acting in the axial direction drives the stopper of the bicameral ampoule.

In some preferred embodiments, a mixing device in accordance with the invention comprises a damping means for damping the drive force acting on the stopper in the axial direction from the drive mechanism. If the drive force is exerted on the stopper of the bicameral ampoule by the drive mechanism is too high to guarantee slow mixing within the ampoule, the translational movement of the drive mechanism can be braked or delayed by the damping means, such that the stopper experiences a smaller drive force and is shifted slowly within the ampoule. In some embodiments, a damping means in accordance with the invention is preferably provided in or associated with the casing of the mixing device. In principle, however, it is also possible to provide a damping means within the bicameral ampoule.

In one preferred embodiment of the present invention, a helical spring is used as the drive mechanism and drives an advancing member for advancing the stopper of the bicameral ampoule. The helical spring and the advancing member are accommodated within a main casing of the mixing device. A sleeve sealed at one end serves as the advancing member and is mounted such that it can be shifted with respect to the main casing. The spring is supported at an end of the casing which opposes the end from which the product is administered. The other end of the spring feeds into the sleeve. The sealed end of the sleeve thus forms an abutment for the helical spring. A casing part can be used as a biasing mechanism and is provided to accommodate the bicameral ampoule and can be detached from the main casing. Before the bicameral ampoule is inserted into the casing part, the casing part can be slid into the main casing of the mixing device via a facing area, wherein it shifts the sleeve within the main casing and compresses the helical spring. In a position of the sleeve in which the spring is compressed, the sleeve cooperates with a locking or latching mechanism, such that the spring is fixed in a biased position.

The casing part serving as the biasing mechanism is removed from the main casing after biasing and the bicameral ampoule is inserted at the open end of the casing part opposite the facing side. The casing part is then attached to the main casing via the open end, such that the stopper of the bicameral ampoule comes to rest opposite the closed end of the sleeve, fixedly connected to the main casing. The mixing device is then in a biased state with an inserted bicameral ampoule and is ready for mixing the injectable product. To this end, the latching of the sleeve is released by the trigger, such that the helical spring performs a translational movement due to its bias and can act on the stopper within the bicameral ampoule via the sleeve. The helical spring is designed with an appropriate spring component for reliably and for slowly mixing the components within the bicameral ampoule.

Once mixed, the injectable product can be administered, for example, by means of a typical administering device. The mixing device of the present invention can advantageously also be formed as part of an administering device.

The mixing device as set forth in the present invention can mix an injectable product in a bicameral ampoule in a simple and uncomplicated way. Only a few hand operations are required in order to enable the components in the ampoule to be reliably mixed. This simplifies the mixing of products, such as growth hormones, in bicameral ampoules.

If the dimensions of the helical spring have not already been designed for slowly performing the mixing procedure, a damping means for damping the drive force exerted on the stopper of the bicameral ampoule by the spring can be provided in accordance with the invention. The damping means can, for example, consist of at least two frictional elements acting against each other. In one embodiment, the frictional elements can be provided by an inner area of the main casing of the mixing device and by an engaging area of the advancing member or sleeve which abuts said inner area. The inner circumference of the casing and the engaging area of the sleeve fit each other such that the engaging area of the sleeve is pressed against the inner area of the casing, whereby a frictional force between the inner area and the engaging area has to be overcome when shifting the sleeve with respect to the casing. The frictional force can be adapted to a desired damping using the surface structure of the areas or a surface coating. It is also possible to configure the frictional force in accordance with a damping by shaping the inner area of the casing. It is, for example, possible to form the inner diameter, i.e., the inner area, of the casing conically diverging in the direction of the advancing direction of the stopper. This constantly reduces the frictional resistance as the advancing member is advanced and the spring force, weakening in the course of advancing, can be equalized. Using such measures, it is therefore possible drive the stopper within the bicameral ampoule at different speeds in the course of advancing.

In another embodiment, the drive mechanism can comprise a memory metal (e.g., nitinol, etc.). In one embodiment, the memory metal element is deformed by the biasing mechanism, such as the casing part described above, and biased. Once a latching mechanism for holding the bias has been released, the memory metal element returns to its original shape, wherein it can exert a drive force on the stopper in the bicameral ampoule. By selecting a suitable metal, the drive force can be selected in accordance with a desired drive speed for the stopper, for mixing. With the aid of the memory metal, it is possible to generate the same drive force over the entire advancing path of the advancing member. This ensures that the stopper is advanced at a constant speed. In order to regulate the drive force acting on the stopper, a damping means comprising, for example, two frictional elements as described above can additionally be used. In some embodiments, a spring made of an appropriate memory metal is preferably used as the memory metal element.

One preferred embodiment of a damping means as set forth in the present invention comprises an air-tight chamber and a reflux valve. The air-tight chamber can, for example, be provided by a space resulting between the sleeve for accommodating the helical spring and the inner area of the casing. Preferably, a cylindrical shell area is arranged coaxially within the casing, wherein the space between the inner area of the casing and the outer area of the cylindrical shell is sealed on one side by a facing side of the sleeve for accommodating the helical spring. On the opposite side, i.e., at the end of the mixing device, one or more reflux valves can be arranged which seal off the space on this side. When the drive mechanism is biased, i.e., when the sleeve is slid into the interior of the casing, the air in the air-tight chamber is forced out of the chamber through the reflux valves by advancing the sealing facing sides of the sleeve. In the biased position, the volume of the chamber is minimized. The reflux valves form a locking mechanism for locking the drive mechanism in the biased position, since no air can penetrate into the chamber when the valve is closed. This holds the spring in its biased position. In order to trigger the drive mechanism, i.e., to transfer the drive force onto the stopper, air can be let into the chamber in doses or increments by the reflux valves. The speed of advancing the stopper within the bicameral ampoule can be controlled by regulating the speed of air penetrating into the air-tight chamber. In this way, slow mixing can be guaranteed. In addition to locking using a reflux valve, a latching mechanism for latching the drive mechanism in the biasing position can be provided. Additionally, a damping means such as is described above can also be provided for further damping the drive force.

In another embodiment of a mixing device as set forth in the present invention, a drive mechanism comprises a vacuum chamber and an air-tight chamber with at least one reflux valve. The vacuum chamber and the air-tight chamber are sealed off from each other by a drive member which can be moved in the axial direction with respect to the casing. In this embodiment, a spring is not needed to generate a bias of the drive mechanism. The vacuum chamber is arranged within the casing of the mixing device and sealed off tightly against the environment. By biasing the drive mechanism, i.e., by shifting a sleeve within the casing of the mixing device, a partial vacuum is generated in the vacuum chamber. The bias simultaneously forces air out of the air-tight chamber through the reflux valve. By closing the valve, the mixing device is in a biased position. In order to mix the fluid product, the reflux valves can let air into the air-tight chamber in doses, such that the drive mechanism is moved in the axial direction due to the equalizing partial vacuum in the vacuum chamber.

The present invention has been explained on the basis of a number of exemplary embodiments. The individual means of a mixing device in accordance with the invention can also be expediently combined with each other. A drive mechanism with a vacuum chamber can, for example, additionally comprise a frictional means and/or a helical spring.

### BRIEF DESCRIPTION OF THE DRAWINGS

FIG. 1 is a longitudinal section through an administering device as set forth in an embodiment of the present invention, in a relaxed state;

FIG. 2 is a longitudinal section through an administering device as set forth in FIG. 1, when biasing the administering device;

FIG. 3 is a longitudinal section through an administering device as set forth in FIG. 1, in a biased state, with an inserted bicameral ampoule; and

FIG. 4 is a longitudinal section through an administering device as set forth in FIG. 1, in a state with a mixed injectable product.

### DETAILED DESCRIPTION

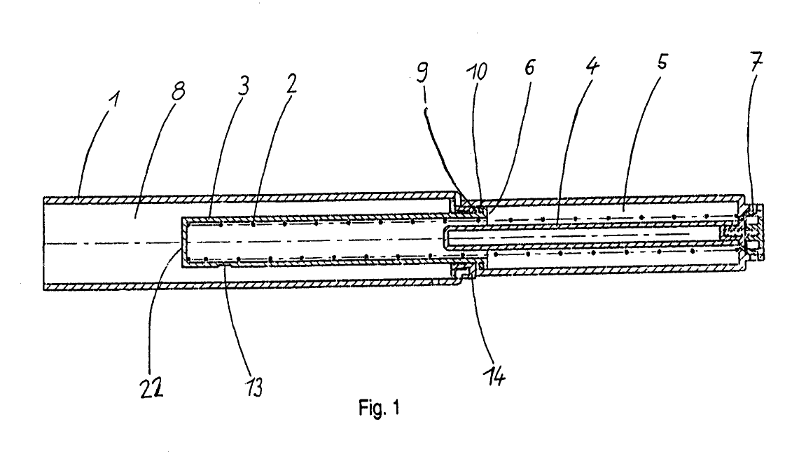

FIG. 1 shows a main casing 1 of a mixing device in accordance with the invention, in which a helical spring 2 and a generally cylindrical and/or tubular spring sleeve 3 mounted such that it can be shifted with respect to the main casing are accommodated. The helical spring 2 is arranged within the spring sleeve 3 and is supported against a closing wall 22 of the spring sleeve 3. The other end of the helical spring 2 is supported against a sealed end of the main casing 1. In this embodiment of the invention, the drive mechanism is formed by the helical spring 2 and the spring sleeve 3. Furthermore, a cylinder 4 is arranged within the main casing 1, generally coaxially with respect to the casing, about which the helical spring 2 comes to rest. Thus, in one embodiment, the casing 1, the spring sleeve 3, the cylinder 4 and the spring 2 are generally coaxially and concentrically arranged.

The mixing device comprises an air-tight chamber 5 which is defined by the inner area of the main casing 1, the inner area of the spring sleeve 3 and the outer area of the cylinder 4. Facing areas 6 on the open side of the spring sleeve 3 connect air-tight to the inner area of the main casing 1. Two reflux valves 7 are provided on the main casing 1 at its sealed end and can establish a connection between the air-tight chamber and the atmosphere. Any suitable valve or flow control structure, including those known to those skilled in the art, may be used.

In FIG. 1, in the relaxed state of the helical spring 2, the spring sleeve 3 protrudes into a space 8 provided for a bicameral ampoule. In this state, the air-tight chamber 5 exhibits a maximum volume. On the outer circumference of the spring sleeve 3, an elevation, raised area or ring 9 is provided at the end with the facing areas 6, the outer area of said elevation or ring forming an engaging area 10 via which the spring sleeve 3 abuts the inner area of the main casing 1. The engaging area 10 and the inner area of the main casing 1 can cooperate such that a frictional force has to be overcome in order to shift the spring sleeve 3 in the axial direction with respect to the main casing 1 and the air-tight chamber 5 is simultaneously sealed tightly when shifting.

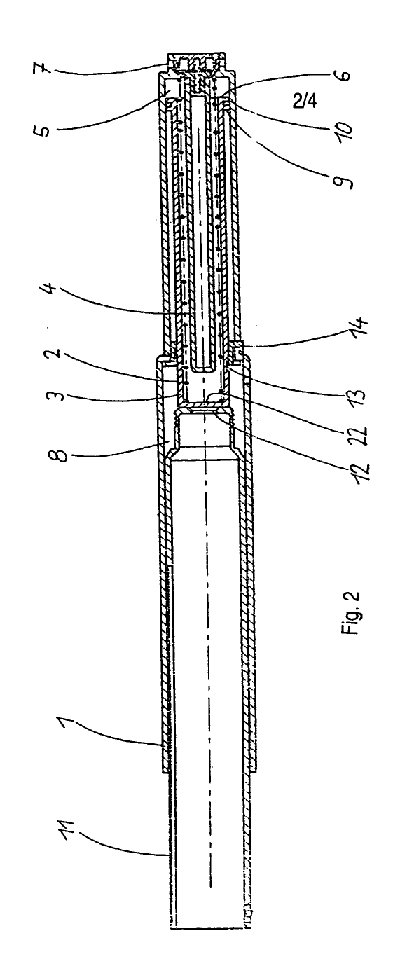

In FIG. 2, the mixing device shown in FIG. 1 is biased using a biasing mechanism. A casing part 11 which can be inserted into the main casing 1 serves as the biasing mechanism and serves to accommodate a bicameral ampoule. The casing part 11 is open at one end and at the other end comprises a facing area 12 against which the bicameral ampoule can be supported and through which an injection needle (not shown) for administering a fluid product can be placed. The casing part 11 for biasing the helical spring 2 is inserted into the space 8 of the main casing 1 via the facing area 12 until it pushes against the closing wall 22 of the spring sleeve 3. By sliding the casing part 11 further into the main casing 1, the spring sleeve 3 is shifted in the axial direction with respect to the main casing 1. This reduces the volume of the air-tight chamber 5 and the forced air escapes from the air-tight chamber 5 through the reflux valves 7.

On the outer circumferential area of the spring sleeve 3, a recess 13 is provided in a region near the closing wall 22 of the spring sleeve 3 and an engaging element 14 is provided in a middle region of the main casing 1, one end of said engaging element protruding laterally out of the main casing 1 and the other end protruding into the main casing as a protrusion. When the helical spring 2 is compressed, i.e., when the spring sleeve 3 is in a position in which it has been slid into the main casing 1, the engaging element 14 engages with the recess 13 of the spring sleeve 3 and latches the spring sleeve 3 to the main casing 1. In this biased state, the drive mechanism—i.e., the helical spring 2 and the spring sleeve 3—is locked by closing the reflux valve 7 and latching in the engaging mechanism 14.

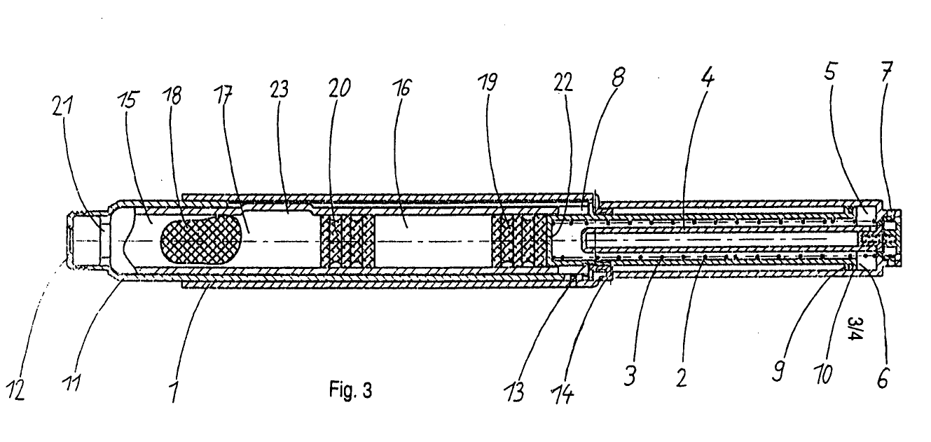

FIG. 3 shows the mixing device with an inserted bicameral ampoule 15. The bicameral ampoule comprises a first chamber 16 with a first product component in the form of a liquid solvent, and a second chamber 17 with a second product component 18 in a solid state. The first chamber 16 is defined by a first stopper 19 and a second stopper 20, wherein the stoppers 19 and 20 can be shifted in the axial direction with respect to the bicameral ampoule. The second chamber 17 is defined by the stopper 20 and a sealed outlet 21. In this position, the stopper 19 abuts the closure 22 of the spring sleeve 3.

In order to insert the bicameral ampoule 15, the casing part 11 is removed from the main casing 1, the bicameral ampoule 15 is slid into the casing part 11 at the open end, until it abuts the facing area 12 from within, and the casing part 11 is inserted into the main casing 1 via the open end. The bicameral ampoule 15 is fixed in the casing part 11 and the casing part 11 is fixedly connected to the main casing 1.

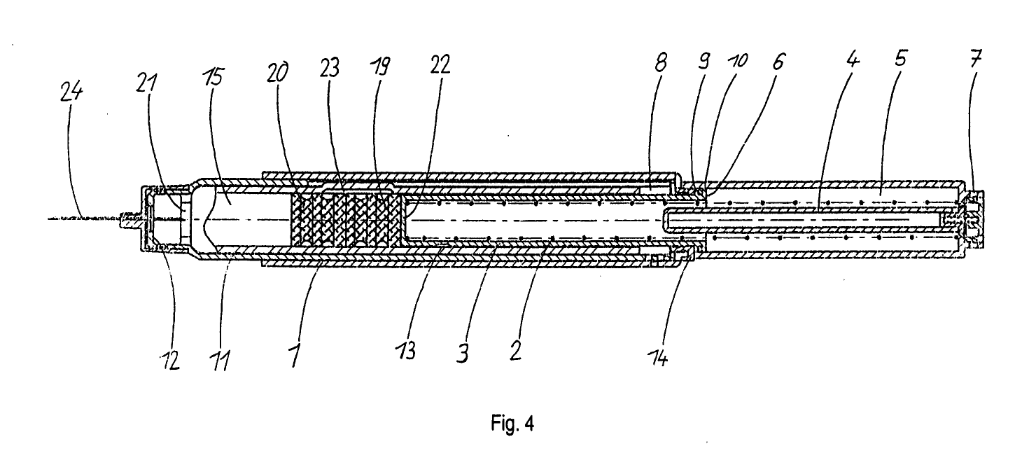

FIG. 4 shows the mixing device when the fluid product is mixed. In order to mix the product components in the bicameral ampoule 15, the engaging element 14 is released from its engagement with the recess 13 on the spring sleeve 3. Air is let into the air-tight chamber 5 via the reflux valve 7, such that the air-tight chamber 5 can expand due to the biasing force of the helical spring 2, i.e., the helical spring 2 exerts a drive force on the spring sleeve 3. In this way, the drive mechanism performs a translational movement and drives the stopper 19 of the bicameral ampoule in the axial direction. In this way, air can be let into the air-tight chamber 5 through the reflux valves 7 in doses, defined or incremental amounts, such that the biasing force of the spring 2 acts on the stopper 19 in a damped or braked way and slow mixing within the bicameral ampoule 15 is achieved.

During mixing, the advancing stopper 19 acts on the stopper 20 via the solvent 16 until the stopper 20 assumes a position within the bicameral ampoule 15, in which the solvent enters the chamber 17 from the chamber 16 via a by-pass 23 and dissolves the solid product component 18.

In the position shown in FIG. 4, the injectable product is, for example, ready to be administered. The mixed product can be administered by an administering mechanism, through the outlet 21 via an injection needle 24. Once the bicameral ampoule 15 has been emptied, the casing part 11 together with the ampoule can be removed from the main casing 1. The mixing device is then ready to accommodate another ampoule.

In an injection device and/or a mixing device as set forth in the present invention, various combinations of drive mechanisms, locking mechanisms, biasing mechanisms or damping means may be combined and/or implemented; such combinations or implementations are intended to form part of the scope of the invention. When combining individual features or means, care need only be taken that the drive mechanism for advancing the stopper 19 can perform a translational movement.

In the foregoing description, embodiments and features of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for mixing an injectable product, comprising:
a) a casing; and
b) an ampoule which comprises at least a first chamber containing a first product component and at least a second chamber containing a second product component, wherein said mixing device comprises a drive mechanism which drives a stopper in the ampoule using a translational movement.

2. The mixing device as set forth in claim 1, wherein the ampoule is fixed with respect to said casing during mixing and said stopper can be driven in the axial direction relative to the casing by said drive mechanism.

3. The mixing device as set forth in claim 1, wherein the drive mechanism drives the stopper automatically.

4. The mixing device as set forth in claim 1, comprising a biasing mechanism for biasing the drive mechanism.

5. The mixing device as set forth in claim 1, wherein the drive mechanism comprises a helical spring.

6. The mixing device as set forth in claim 1, wherein the drive mechanism comprises a memory metal.

7. The mixing device as set forth in claim 1, wherein a lock means is provided for locking the drive mechanism in a biased position.

8. The mixing device as set forth in claim 1, wherein a damping means is provided for damping the drive force acting on the stopper from the drive mechanism.

9. The mixing device as set forth in claim 8, wherein said damping means comprises at least two frictional elements acting against each other.

10. The mixing device as set forth in claim 8, wherein the damping means comprises an air-tight chamber connected to the drive mechanism and at least one valve associated with said chamber.

11. The mixing device as set forth in claim 1, wherein the drive mechanism comprises a vacuum chamber operably coupled to an air-tight chamber via a valve.

12. An administering device for administering an injectable product from a multiple-chamber ampoule, comprising a mixing device comprising:
a casing;
drive means for exerting a driving force against a stopper in the ampoule; and
damping means for reducing the driving force.
